SEQUENCE LISTING (17 sequences)

SEQ ID NO: 1 - protein, 8 aa, Artificial Sequence
  Synthetic polypeptide of a FLAG tag epitope
DYKDDDDK

SEQ ID NO: 2 - protein, 9 aa, Artificial Sequence
  Synthetic polypeptide of a HA tag
YPYDVPDYA

SEQ ID NO: 3 - protein, 9 aa, Artificial Sequence
  Synthetic polypeptide of a linker sequence
GGSDDVQAE

SEQ ID NO: 4 - protein, 10 aa, Artificial Sequence
  Synthetic polypeptide of a Myc tag
EQKLISEEDL

SEQ ID NO: 5 - protein, 23 aa, Homo sapiens
MDAMKRGLCCVLLLCGAVFVSPS

SEQ ID NO: 6 - DNA, 9 bp, Unknown
  Synthetic oligonucleotide
gccgccacc

SEQ ID NO: 7 - DNA, 19 bp, Artificial Sequence
  Synthetic oligonucleotide
cacttctggtgcttctggc

SEQ ID NO: 8 - DNA, 19 bp, Artificial Sequence
  Synthetic oligonucleotide
aagatccgctacttgctcc

SEQ ID NO: 9 - protein, 50 aa, Artificial Sequence
  Synthetic construct
RWKVVVISAILALVVLTVISLIILIMLWGSDYKDDDDKGMQYPYDVPDYA

SEQ ID NO: 10 - protein, 50 aa, Artificial Sequence
  Synthetic construct
RWKVVVISAILALVVLTVISLIILIMLWGSDTKEDEDRGMQYPYDVPDYA

SEQ ID NO: 11 - protein, 49 aa, Artificial Sequence
  Synthetic construct
RWKVVVISAILALVVLTVISLIILIMLWGSDTKEDEDRGMQDYKDDDDK

SEQ ID NO: 12 - protein, 49 aa, Artificial Sequence
  Synthetic construct
WKVVVISAILALVVLTVISLIILIMLWGSEYRDEDEKGMQYPYDVPDYA

SEQ ID NO: 13 - protein, 48 aa, Artificial Sequence
  Synthetic construct
WKVVVISAILALVVLTVISLIILIMLWGSEYRDEDEKGMQDYKDDDDK

SEQ ID NO: 14 - protein, 48 aa, Artificial Sequence
  Synthetic construct
WKVVVISAILALVVLTVISLIILIMLWGSGGSDDVQAEEQKLISEEDL

SEQ ID NO: 15 - protein, 70 aa, Artificial Sequence
  Synthetic construct
WKVVVISAILALVVLTVISLIILIMLWGSDYKDDDDKGMQFNNFTVSFWLRVPKVSASHLEYPYDVPDYA

SEQ ID NO: 16 - protein, 74 aa, Artificial Sequence
  Synthetic construct
WKVVVISAILALVVLTVISLIILIMLWGSEYRDEDEKGMQAENKFNNFTVSFWLRVPKVSASHLEYPYDVPDYA

SEQ ID NO: 17 - protein, 66 aa, Artificial Sequence
  Synthetic construct
WKVVVISAILALVVLTVISLIILIMLWGSEYRDEDEKGMQAENKAKFVAAWTLKAAAYPYDVPDYA

(19) United States
(12) Patent Application Publication
(10) Pub. No.: US 2012/0231474 A1
(43) Pub. Date: Sep. 13, 2012

(54) CONSTRUCTS THAT ALLOW FOR DETECTION AND QUANTITATION OF MEMBRANE PROTEINS

(76) Inventors: Ross Stewart Chambers, Hockessin, DE (US); Michael Craig Brown, North East, MD (US); James William Stave, Bear, DE (US)

(21) Appl. No.: 13/402,423

(22) Filed: Feb. 22, 2012

Related U.S. Application Data
(60) Provisional application No. 61/445,220, filed on Feb. 22, 2011.

(51) Int. Cl. G01N 33/577; C07K 19/00; C12P 21/02; C12N 15/63

(52) U.S. Cl. 435/7.2; 435/320.1; 530/350; 435/69.7

(57) ABSTRACT

A construct is described that allows for detection and quantitation of membrane-bound polypeptides.

### CROSS REFERENCE TO RELATED APPLICATIONS

This application claims the benefit of priority under 35 U.S.C. 119(e) to U.S. Application No. 61/445,220, filed on Feb. 22, 2011.

### TECHNICAL FIELD

This disclosure generally relates to detection and quantitation of membrane proteins.

### BACKGROUND

Measuring the amount of membrane protein on the surface of cells is important to understand trafficking mechanisms and to allow optimization of surface expression levels. Membrane proteins often can be localized to internal pools in cells, either due to trafficking mechanisms or trapping of the protein in the endoplasmic reticulum due to mis-folding and aggregation.

A construct is described herein that allows for detecting and measuring membrane proteins on the surface of a cell.

### SUMMARY

A construct is described that allows for detection and quantitation of membrane-bound polypeptides.

In one aspect, a nucleic acid construct is provided. Such a construct typically includes a nucleic acid encoding a membrane protein operably linked to a nucleic acid encoding a single membrane-spanning domain operably linked to a nucleic acid encoding a detectable protein, wherein the C-terminus of the membrane protein is cytoplasmic; and wherein the single membrane-spanning domain is configured such that the N-terminus of the membrane-spanning domain is intracellular and the C-terminus of the membrane-spanning domain is extracellular. In certain embodiments, the membrane protein is a G-protein coupled receptor (GPCR) protein, an ion channel protein, or a single pass type I membrane protein. In certain embodiments, the single membrane-spanning domain is from a protein selected from the group consisting of PDGFR B. In certain embodiments, the detectable protein is selected from the group consisting of FLAG, HA, and Myc.

Representative ion channel proteins include potassium channel proteins, TRP channel proteins, four-domain channel proteins, chloride channel proteins, ligand-gated ion channel proteins, transporter proteins, CD marker proteins, tetraspannin proteins, and aquaporin proteins. Representative membrane proteins include VIPR1, ADORA2A, F2R, EP4, CXCR4, LPAR1, GRPR, ADRB2, EAG, Nav1.7, CLCA1, nAChR, ABCA1, SLC5A1, MS4A1, AQP1, CD33, CD20, DARC, Kir2.1, Kir2.2, Kir7.1, Kv10.1, Kv11.1, TASK3, TRPV3, and TRPV3. In a representative construct, the membrane protein is CD20, DARC, or CXCR4; the single membrane-spanning domain is from PDGFRB; and the detectable protein is HA or FLAG.

In another aspect, a construct for detecting a membrane-bound protein is provided. Such a construct typically includes, in the 5′ to 3′ direction, a multiple cloning site (MCS) for in-frame cloning of a nucleic acid encoding a membrane protein, operably linked to a nucleic acid encoding a single membrane-spanning domain, wherein the single membrane-spanning domain is configured such that its N-terminus is intracellular and its C-terminus is extracellular, operably linked to a nucleic acid encoding a detectable protein.

Such a construct further can include a promoter that is operably linked to the 5′ end of the MCS. A representative single membrane-spanning domain is from a protein selected from the group consisting of PDGFR B. A representative detectable protein is FLAG, HA, or Myc. In certain embodiments, the membrane protein is CD20, DARC, or CXCR4; the single membrane-spanning domain is from PDGFRB; and the detectable protein is HA or FLAG.

In still another aspect, a nucleic acid construct is provided. Such a construct typically includes a nucleic acid encoding a membrane protein operably linked to a nucleic acid encoding a single membrane-spanning domain operably linked to a nucleic acid encoding a detectable protein, wherein the C-terminus of the membrane protein is cytoplasmic; and wherein the single membrane-spanning domain is configured such that the N-terminus of the membrane-spanning domain is intracellular and the C-terminus of the membrane-spanning domain is extracellular. In various embodiments, the membrane protein is a G-protein coupled receptor (GPCR) protein, an ion channel protein, or a single pass type I membrane protein.

In yet another aspect, a fusion protein is provided. Typically, the fusion protein includes, in the amino- to carboxy-terminal direction, a membrane protein having a cytoplasmic C-terminus, a single membrane-spanning domain having an extracellular C-terminus, and a detectable protein. In certain embodiments, the membrane protein is a G-protein coupled receptor (GPCR) protein, an ion channel protein, or a single pass type I membrane protein. In certain embodiments, the single membrane-spanning domain is from a protein selected from the group consisting of PDGFR B. In certain embodiments, the detectable protein is selected from the group consisting of FLAG, HA, and Myc. In certain instances, the single membrane-spanning domain is heterologous to the membrane protein.

Representative ion channel proteins include potassium channel proteins, TRP channel proteins, four-domain channel proteins, chloride channel proteins, ligand-gated ion channel proteins, transporter proteins, CD marker proteins, tetraspannin proteins, and aquaporin proteins. Representative membrane proteins include VIPR1, ADORA2A, F2R, EP4, CXCR4, LPAR1, GRPR, ADRB2, EAG, Nav1.7, CLCA1, nAChR, ABCA1, SLC5A1, MS4A1, AQP1, CD33, CD20, DARC, Kir2.1, Kir2.2, Kir7.1, Kv10.1, Kv11.1, TASK3, TRPV3, and TRPV3. In certain embodiments, the membrane protein is CD20, DARC, or CXCR4; the single membrane-spanning domain is from PDGFRB; and the detectable protein is HA or FLAG.

In still another aspect, a method of expressing a fusion protein is provided. Such a method typically includes the steps of culturing a recombinant host cell comprising the construct of claim 1 under conditions that promote the expression of the fusion polypeptide.

In yet another aspect, a method of detecting a membrane protein bound to the membrane of a cell is provided. Such a method typically includes the steps of (a) culturing a recombinant host cell that comprises a nucleic acid molecule that encodes the fusion protein of claim 14 under conditions suitable for expression of the fusion polypeptide; and (b) detecting the detectable protein portion of the fusion protein bound to the cell, thereby detecting the membrane protein bound to the membrane of the cell. In certain embodiments, the host cell is a eukaryotic host cell. Representative eukaryotic host cells include HEK-293 cells, CHO cells, Swiss 3T3 cells, and yeast cells. In certain embodiments, the detecting step utilizes a labeled antibody that binds to the detectable protein.

In yet another aspect, a method of quantitating the amount of a membrane protein bound to the membrane of a cell is provided. Such a method typically includes the steps of (a) culturing a recombinant host cell that comprises a nucleic acid molecule that encodes the fusion protein of claim 14 under conditions suitable for expression of the fusion polypeptide; and (b) quantitating the amount of detectable protein bound to the cell, thereby quantitating the amount of the membrane protein bound to the membrane of the cell. In certain embodiments, the host cell is a eukaryotic host cell. Representative eukaryotic host cells include HEK-293 cells, CHO cells, Swiss 3T3 cells, and yeast cells. In certain embodiments, the quantitating step comprises contacting the cell with a labeled antibody that binds to the detectable protein and determining the amount of labeled antibody that bound.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIG. 1 shows a schematic of the snorkel tag. A representative four span multipass membrane protein is shown (black) fused to a transmembrane domain (grey) and an epitope tag.

FIG. 2 shows the correlation between membrane protein and snorkel tag expression. Light grey lines indicate untransfected control cells; dark grey lines indicate the transfected cells. Panel A shows data for cells transfected with CD20 cloned into pSNKL-Q stained with anti-HA and anti-CD20 antibodies; Panel B shows data from the same construct used in Panel A but with a stop codon inserted before the snorkel tag; Panel C shows data from the same construct used in Panel A but containing DARC instead of CD20; Panel D shows data from the same construct used in Panel B but containing DARC instead of CD20; and Panel E shows data from the same construct used in Panel A but containing CXCR4 instead of CD20.

FIG. 3 shows the expression of the snorkel tag with a panel of ten membrane proteins. Cells were first stained with the anti-HA antibody (light grey), permeabilized and restained again with anti-HA antibody (dark grey). Panel A, VIPR1; Panel B, ADORA2A; Panel C, F2R; Panel D, EP4; Panel E, CXCR4; Panel F, LPAR1; Panel G, GRPR; Panel H, ADRB2; Panel I, CD20; and Panel J, DARC.

FIG. 4 shows the effect that multimerization domains have on snorkel tag expression. Unfilled light grey indicates untransfected control cells; filled dark grey indicates the transfected cells. Panels A, B, and C were stained with anti-HA antibodies; panels D, E, F, G, H were stained with anti-CD20 antibodies. Panels A and D, pSNKL-Q construct; Panels B and E, pSNKL construct with a dimerization domain; Panels C and F, pSNKL construct with a tetramerization domain; Panel G, CD20 gene with no snorkel or multimerization domain; Panel H, CD20 gene with no snorkel but with the tetramerization domain.

FIG. 5 shows the effect that multimerization domains have on snorkel tag expression. Unfilled light grey indicates untransfected control cells; filled dark grey indicates the transfected cells. Panels A, B, and C were stained with anti-HA antibodies; panels D, E, F, and G were stained with anti-DARC antibodies. Panels A and D, pSNKL-Q construct; Panels B and E, pSNKL construct with a dimerization domain; Panels C and F, pSNKL construct with a tetramerization domain; and Panel G, DARC gene with no snorkel or multimerization domain.

FIG. 6 shows the effect that tetramerization domains have on different transmembrane domains. The membrane protein genes were cloned into pSNKL with a tetramerization domain either with the PDGFRB or TFR1 domain. Light grey indicates untransfected control cells. Panel A, CD20; Panel B, CD33; and Panel C, DARC.

FIG. 7 shows the use of the Myc tag in the snorkel. The ADORA2A gene was cloned into pSNKL-M, which contains the Myc tag instead of the HA tag. Black represents the untransfected control cells; grey represents the transfected cells. Cells were first stained with the anti-Myc antibody (Panel A), then permeabilized and re-stained with the anti-Myc antibody (Panel B).

FIG. 8 shows ion channel expression in pSNKL-UH3. The “snorkel tag” was evaluated using ion channels, CD20, TASK3, Kir2.1, Kv1.3 and KCa3.1.

FIG. 9 shows the expression of ADORA2A in various “snorkel constructs.” SEQ ID NOs: 9-17 (top to bottom).

FIG. 10A and FIG. 10B shows the expression of CD20 in various “snorkel constructs.” SEQ ID NOs: 9-17 (FIG. 10A, top to bottom).

Like reference symbols in the various drawings indicate like elements.

### DETAILED DESCRIPTION

This disclosure describes “snorkel constructs” and methods of using such constructs for expressing and/or detecting a membrane protein on the surface of a cell. One of the advantages of the “snorkel construct” is that the detectable protein portion of the “snorkel construct” (e.g., an epitope tag) is not attached to one of the extracellular regions of the membrane protein. Therefore, there is less likelihood that the structure and/or function of the membrane protein will be perturbed when using the “snorkel construct.” This technology is particularly important for membrane proteins such as G-protein-coupled receptors (GPCRs) and ion channel proteins, which often have only small loops exposed extracellularly.

### “Snorkel” Nucleic Acids and Polypeptides

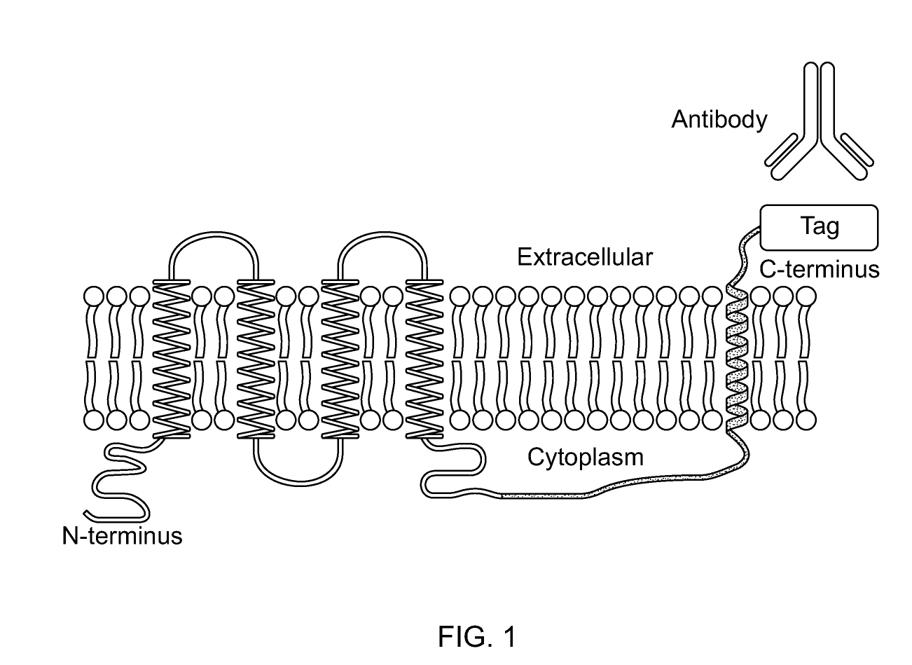

Nucleic acid constructs are described herein, referred to as “snorkel constructs,” that encode a fusion protein. A “snorkel construct” as described herein includes a nucleic acid encoding a membrane protein, which is operably linked to a nucleic acid encoding a single membrane-spanning domain, which is, in turn, operably linked to a nucleic acid encoding a detectable protein. When expressed, such a “snorkel construct” produces a fusion protein that includes, in the amino- to carboxy-terminal direction, a membrane protein having a cytoplasmic C-terminus, a single membrane-spanning domain having an extracellular C-terminus, and a detectable protein. See, for example, FIG. 1.

Membrane proteins, and the nucleic acids encoding them, number in the thousands and have been well documented in the literature. A number of classes of membrane proteins are recognized based on the topology of the membrane protein or segments therein (e.g., extracellular, intracellular, the number of times the membrane protein traverses the membrane), the sequence identity of genes and/or the encoded proteins, and/or the function of the protein. Representative classes of membrane proteins include, without limitation, G-protein coupled receptor (GPCR) proteins, ion channel proteins, or single pass type I membrane proteins.

Representative GPCR proteins include, for example, ADRB2, VIPR1, ADORA2A, F2R, LPAR1, GRPR, EP4, CXCR4, and DARC. Within the ion channel proteins, there are a number of subclasses. For example, ion channel proteins can be potassium channel proteins (e.g., EAG (also known as Kv10.1), Kir2.1, Kir2.2, Kir7.1, Kv11.1, TASK3), TRP channel proteins (e.g., TRPV3), four-domain channel proteins (e.g., Nav1.7), chloride channel proteins (e.g., CLCA1), ligand-gated ion channel proteins (e.g., nAChR), transporter proteins (e.g., ABCA1, SLC5A1), CD marker proteins (e.g., CD33, CD20), tetraspannin proteins (e.g., MS4A1), and aquaporin proteins (e.g., AQP1). The membrane proteins specifically called out in this paragraph are meant to be representative and the list is not exhaustive. Those of skill in the art would understand that the “snorkel construct” is particularly well-suited for membrane proteins that have a cytoplasmically located C-terminus (e.g., type I, type III, type IV, or type VI membrane proteins).

As used herein, a single membrane-spanning domain refers to polypeptide sequence that spans a cell membrane a single time. For the “snorkel construct,” it is desired that the single membrane-spanning domain be configured such that the N-terminus of the membrane-spanning domain is intracellular and the C-terminus of the membrane-spanning domain is extracellular. A representative single membrane-spanning domain is from a PDGFR B protein. Although the membrane-spanning PDGFR B domain used herein was genetically engineered to adopt the desired conformation (i.e., intracellular N-terminus, extracellular C-terminus), single membrane-spanning domains from other proteins that naturally have the desired configuration also can be used. Typically, the single membrane-spanning domain is from a polypeptide that is heterologous to the membrane protein.

As used herein, detectable proteins include, for example, any type of protein that can be detected. A number of detectable proteins are known and routinely used in the art as tags including, without limitation, the FLAG tag (see, for example, U.S. Pat. No. 4,703,004), the hemagglutinin (HA) tag, the myc-tag, and 6× His tag. See, also, Terpe, 2003, Appl. Microbiol. Biotechnol., 60:523-33. Although these examples of epitope tags are provided herein, those of skill in the art would understand that any protein that can be detected is suitable for use in a “snorkel construct.”

Operably linked, as used herein, refers to an in-frame junction between coding sequences such that a correct (i.e., in frame) polypeptide is produced following transcription and translation of the sequences. With respect to promoter and/or other regulatory element(s), operably linked means that such sequences are positioned in a construct relative to a coding sequence in such a way as to direct or regulate expression of the coding sequence. Elements necessary for expression include nucleic acid sequences that direct and regulate expression of coding sequences. Elements necessary for expression can include promoters, introns, enhancer sequences, termination sequences, response elements, or inducible elements that modulate expression of a nucleic acid. Elements necessary for expression can be native to the membrane protein or can be heterologous to the membrane protein. Heterologous sequences can be of bacterial, yeast, insect, mammalian, or viral origin and constructs can contain a combination of elements from different origins.

In certain embodiments, a “snorkel construct” may be produced that contains a “multiple cloning site” (MCS), sometimes referred to as a “polylinker,” instead of a particular membrane protein positioned 5′ of the nucleic acid encoding the single membrane-spanning domain. In this type of construct, the MCS would allow the “snorkel construct” to be used with any number of different membrane proteins. As used herein, a MCS refers to nucleic acid sequences containing at least one, and usually several, consensus sites for different restriction enzymes. Those skilled in the art will appreciate the various features that MCSs can offer such as, without limitation, the multitude of cloning options as well as different reading frame positions. It also will be appreciated by those skilled in the art that “restriction enzymes” typically refer to endonuclease enzymes that cleave double-stranded DNA at or near a specific consensus sequence. A “snorkel construct” for expressing a membrane protein additionally can include sequences such as those encoding a selectable marker (e.g., an antibiotic resistance gene), and/or those that can be used in purification of a polypeptide (e.g., 6× His tag).

As used herein, nucleic acid molecules can be DNA or RNA, linear or circular, and in sense or antisense orientation. “Isolated,” as used herein, refers to a nucleic acid molecule corresponding to part or all of a gene encoding a polypeptide, but free of sequences that normally flank one or both sides of the wild-type gene in a naturally occurring genome. The term “isolated” also includes any non-naturally-occurring nucleic acid sequence. An isolated nucleic acid includes, for example, a DNA molecule, provided one of the nucleic acid sequences normally found immediately flanking that DNA molecule in a naturally-occurring genome is removed or absent. Thus, an isolated nucleic acid molecule includes, without limitation, a DNA molecule that exists as a separate molecule (e.g., a cDNA or genomic DNA fragment produced by PCR or restriction endonuclease digestion) independent of other sequences, as well as DNA that is incorporated into a vector, an autonomously replicating plasmid, or a virus (e.g., a retrovirus, adenovirus, or herpes virus). In addition, an isolated nucleic acid can include a recombinant DNA molecule that is part of a hybrid or fusion nucleic acid molecule. A nucleic acid molecule existing among hundreds to millions of other nucleic acid molecules within, for example, a nucleic acid library (e.g., a cDNA, or genomic library) or a portion of a gel (e.g., agarose, or polyacrylamine) containing restriction-digested genomic DNA is not to be considered an isolated nucleic acid.

“Purified” as used herein refers to a polypeptide that has been separated from at least some of those components that naturally accompany it. A polypeptide is purified when it is at least 50%, by weight, free from the polypeptides and other naturally occurring organic molecules with which it is naturally associated in vivo. For example, a polypeptide is purified if it is at least 60%, 70%, 75%, 80%, 85%, 90%, 95%, or 99%, by weight, free from other polypeptides. A purified polypeptide can be obtained, for example, by extraction from a natural source; by in vitro or in vivo expression of a recombinant nucleic acid; or by chemically synthesizing a polypeptide. Polypeptides also can be purified using, for example, immunoaffinity chromatography and an antibody, polyacrylamide gel electrophoresis, or HPLC analysis.

The nucleic acids encoding the proteins used in a “snorkel construct” as described herein can be identical to wild type sequences, or they can deviate from the sequence of the wild type nucleic acid or proteins. For example, in some embodiments, a sequence used in or encoded by a “snorkel construct” can have at least about 75% sequence identity, for example, at least about 80% sequence identity, at least about 90% sequence identity, at least about 95% sequence identity, at least about 98% sequence identity, or at least about 99% sequence identity, to a wild type sequence. Percent sequence identity can be determined for any nucleic acid or amino acid sequence relative to a second nucleic acid or amino acid sequence as follows. Briefly, a first nucleic acid or amino acid sequence is compared and aligned to a second nucleic acid or amino acid sequence using BLAST, available at ncbi.nlm.nih.gov on the World Wide Web. The algorithms also are described in detail by Karlin et al. (Proc. Natl. Acad. Sci. USA, 87:2264 (1990) and 90:5873 (1993)) and Altschul et al. (Nucl. Acids Res., 25:3389 (1997)). BLAST can perform a comparison between two sequences using either the BLASTN (to compare nucleic acid sequences) or BLASTP (to compare amino acid sequences) algorithm. If necessary, gaps of one or more residues are inserted into the first or the second sequence to maximize alignments between structurally conserved domains (e.g., alpha-helices, beta-sheets, and loops). Default parameters typically are used when performing sequence alignments.

Once aligned, a length is determined by counting the number of nucleotides or amino acid residues from one sequence that match the other sequence starting with any matched position and ending with any other matched position. A matched position is any position where an identical nucleotide or amino acid residue is present in both the first sequence and the second sequence. The percent identity over a particular length is determined by counting the number of matched positions over that particular length, dividing that number by the length and multiplying the resulting value by 100. It will be appreciated that a known nucleic acid or amino acid sequence that aligns with a subject sequence can result in many different lengths with each length having its own percent identity. While the length value will always be an integer, it is noted that the percent identity value should be rounded to the nearest tenth.

### Methods of Making and Using “Snorkel Constructs”

“Snorkel constructs” as described herein can be produced by recombinant DNA technology methods that are routine in the art. For example, nucleic acids expressing the various components of a “snorkel construct” can be obtained (e.g., isolated) using any method including, without limitation, routine molecular cloning and chemical nucleic acid synthesis techniques. Polymerase chain reaction (PCR) techniques can be used to isolate a nucleic acid molecule. General PCR techniques are described, for example in PCR Primer: A Laboratory Manual, Dieffenbach & Dveksler, Eds., Cold Spring Harbor Laboratory Press, 1995. Furthermore, nucleic acid hybridization techniques can be used to isolate a nucleic acid. Briefly, a nucleic acid sequence encoding a polypeptide can be used as a probe to identify a similar nucleic acid by hybridization under conditions of moderate to high stringency. Moderately stringent hybridization conditions include hybridization at about 42° C. in a hybridization solution containing 25 mM KPO₄ (pH 7.4), 5×.SSC, 5× Denhart's solution, 50 μg/ml denatured, sonicated salmon sperm DNA, 50% formamide, 10% Dextran sulfate, and 1-15 ng/ml probe (about 5×10⁷ cpm/μg), and wash steps at about 50° C. with a wash solution containing 2× SSC and 0.1% SDS. For high stringency, the same hybridization conditions can be used, but washes are performed at about 65° C. with a wash solution containing 0.2× SSC and 0.1% SDS. See, for example, sections 7.39-7.52 of Sambrook, Fritsch & Maniatis, Molecular Cloning: A Laboratory Manual, 2nd Ed., 1989, Cold Spring Harbor Laboratory Press, Plainview, N.Y. Once a nucleic acid is obtained (e.g., isolated), it can then be sequenced and further analyzed using routine methods.

Methods for detecting nucleic acids or the protein products of nucleic acids are known to those of skill in the art. Transcription of nucleic acid sequences can be detected by Northern blotting. Immunoassay formats (e.g., solid-phase immunoassays such as ELISA, and Western blots) can be used to detect a polypeptide and are well known in the art. See, Short Protocols in Molecular Biology, Ch. 11, John Wiley & Sons, Ed., Ausubel et al., 1992. In addition to ELISA and Western blots, solid-phase immunoassays include competition immunoassays, immobilized-antigen immunoassays, immobilized-antibody immunoassays, and double-antibody immunoassays. Further, several types of mass spectrometry (MS) are available and routinely used in the art to detect nucleic acids and proteins, and include immunoprecipitation-mass spectrometry (IP-MS), Fourier-transform MS, ion-trap MS, magnetic-sector MS, quadropole MS and time-of-flight (TOF) MS.

Detection of nucleic acids or polypeptides in vitro or in vivo is usually via a label, e.g., a radioactive label (e.g., ³H, ¹²⁵I, ¹³¹I, ³²P, ³⁵S, and ¹⁴C) or a non-radioactive label (e.g., a fluorescent label, a chemiluminescent label, a paramagnetic label, or an enzyme label) using techniques known to those of ordinary skill in the art. Examples of enzyme labels used routinely in the art for detection and quantitation include horseradish peroxidase (HRP) and alkaline phosphatase (AP). The substrates available for either HRP or AP labels are known in the art and can be selected based upon the desired method of detecting complex formation (e.g., a fluorogenic, chemiluminescent or calorimetric signal).

As described herein, a “snorkel construct” can be introduced into a host cell such that expression occurs and a fusion protein is produced. Many methods for introducing nucleic acids into host cells, whether in vivo or in vitro, are well known to those skilled in the art. For example, calcium phosphate precipitation, electroporation, heat shock, lipofection, microinjection, and viral-mediated nucleic acid transfer are common methods for introducing nucleic acid into host cells. In addition, naked DNA can be delivered directly to host cells in vivo as described elsewhere. See, for example, U.S. Pat. Nos. 5,580,859 and 5,589,466.

Host cells into which “snorkel constructs” are introduced can be prokaryotic host cells or eukaryotic host cells. Representative prokaryotic host cells include, for example, E. coli cells. Representative eukaryotic host cells include, for example, HeLa cells, HEK-293 cells, CHO cells, Swiss 3T3 cells, COS cells, insect cells, or yeast cells (e.g., S. cerevisiae or S. pombe).

A host cell containing a “snorkel construct” can be cultured under appropriate conditions such that expression of the “snorkel construct” takes place and positioning of the membrane protein as well as the single membrane-spanning domain in the cell membrane occurs. Following appropriate positioning of the membrane protein and the single membrane-spanning domain in the membrane, the extracellular detectable protein portion of the “snorkel construct” can be detected. Oftentimes, detection of the detectable protein utilizes a labeled antibody that specifically recognizes and binds to the detectable protein, although the particular methods and reagents will depend upon that particular detectable protein that is used in the “snorkel construct.” In certain instances, it is desirable to quantitate the amount of membrane protein in a cell membrane. The “snorkel construct” allows the amount of membrane protein in the cell membrane to be directly quantitated by measuring the amount of the extracellularly-positioned detectable protein.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1

### “Snorkel Tag” Construction: A Transmembrane Domain and A Tag

A “snorkel tag” was designed using a transmembrane domain (residues 530 to 555) from mouse Beta-type platelet-derived growth factor receptor B (PDGFRB) (Uniprot Accession No. P05622). The sequences naturally flanking the transmembrane domain were altered to conform to the “positive inside” since the topology is reversed (Dowhan et al., 2009, Ann. Rev. Biochem., 78:515-40). The natural C-terminal region of the PDGFRB (residues 556 to 566) was moved to the N-terminus and, at the C-terminus, a short linker (GS), a FLAG tag epitope (DYKDDDDK (SEQ ID NO:1)), a short sequence for a SphI restriction site (encoding residues GMQ), the 9 amino acid HA tag (YPYDVPDYA (SEQ ID NO:2)), and a stop codon were placed. The entire extracellular region of this “snorkel tag” encompasses 22 amino acid residues.

A “snorkel vector” utilizing the PDGFRB transmembrane domain, pSNKL-Q, was constructed by synthesizing a DNA fragment encoding the “snorkel tag” with EcoRI and AgeI restriction sites. The DNA fragment was cloned into the EcoRI and XmaI site of the expression plasmid pCI (Promega; Madison, Wis.). The constructs with a dimerization domain were created from synthetic oligonucleotides and encoded a 44 amino acid residue fragment of a dimeric coiled-coil domain (Zhu et al., 2000, J. Mol. Biol., 300:1377-87) inserted in between the target membrane protein and the “snorkel tag”. The constructs with a tetramerization domain were created from synthetic oligonucleotides and encoded a 105 amino acid residue fragment of a tetrameric coiled-coil domain (Zhiguang et al., 2009, FEBS J., 276:6236-46) inserted in between the target membrane protein and the “snorkel tag”.

A “snorkel vector” utilizing the TFR1 transmembrane domain was designed by substituting the “snorkel tag” described above with residues 53 to 99 (47 amino acid residues) from mouse TFR1 (Uniprot Accession No. Q62351). This region of the mouse TFR1 protein contains a transmembrane domain along with 15 and 11 amino acid residues on the N- and C-terminal ends of the transmembrane domain, respectively. In this construct, the 11 residue C-terminal region flanking the TFR1 transmembrane domain served as the tag. The TFR1 sequence was further modified by C67S, C89S and C98S substitutions to prevent palmitoylation and dimerization (Gosse et al., 2005, J. Immunol., 175:2123-31; Alvarez et al., 1989, EMBO J., 8:2231-40).

A “snorkel tag” utilizing a Myc tag was created by substituting the 22 amino acid residue extracellular portion of the “snorkel tag” described above with a linker (GGSDDVQAE (SEQ ID NO:3)) followed by the Myc tag (EQKLISEEDL (SEQ ID NO:4)).

### Example 2

### Membrane Proteins

The genes encoding the membrane proteins were based on the human sequence and were synthesized from oligonucleotides using a codon table with frequently used mammalian codons.

| Amino | | Usage |
|---|---|---|
| acid | Codon | frequency |
| F | TTT | 0.5 |
| F | TTC | 0.5 |
| L | TTA | 0 |
| L | TTG | 0 |
| L | CTT | 0 |
| L | CTC | 0.25 |
| L | CTA | 0 |
| L | CTG | 0.75 |
| I | ATT | 0.5 |
| I | ATC | 0.5 |
| I | ATA | 0 |
| M | ATG | 1 |
| V | GTT | 0.33 |
| V | GTC | 0.33 |
| V | GTA | 0 |
| V | GTG | 0.34 |
| Y | TAT | 0.5 |
| Y | TAC | 0.5 |
| H | CAT | 0.5 |
| H | CAC | 0.5 |
| Q | CAA | 0.5 |
| Q | CAG | 0.5 |
| N | AAT | 0.5 |
| N | AAC | 0.5 |
| K | AAA | 0.5 |
| K | AAG | 0.5 |
| D | GAT | 0.5 |
| D | GAC | 0.5 |
| S | TCT | 0.33 |
| S | TCC | 0.33 |
| S | TCA | 0 |
| S | TCG | 0 |
| S | AGT | 0 |
| S | AGC | 0.34 |
| P | CCT | 0.5 |
| P | CCC | 0 |
| P | CCA | 0.5 |
| P | CCG | 0 |
| T | ACT | 0.3 |
| T | ACC | 0.4 |
| T | ACA | 0.3 |
| T | ACG | 0 |
| A | GCT | 0.33 |
| A | GCC | 0.33 |
| A | GCA | 0.34 |
| A | GCG | 0 |
| E | GAA | 0.5 |
| E | GAG | 0.5 |
| C | TGT | 0.5 |
| C | TGC | 0.5 |
| W | TGG | 1 |
| R | CGT | 0 |
| R | CGC | 0.25 |
| R | CGA | 0 |
| R | CGG | 0.25 |
| R | AGA | 0.25 |
| R | AGG | 0.25 |
| G | GGT | 0.2 |
| G | GGC | 0.3 |
| G | GGA | 0.25 |
| G | GGG | 0.25 |

With the exception of CD20, all genes had a leader sequence from the human tissue plasminogen activator fused to the N-terminus (MDAMKRGLCC VLLLCGAVFV SPS (SEQ ID NO:5)). A kozak sequence (GCCGCCACC (SEQ ID NO:6)) was added to the 5′ end of the gene and universal primers were added to both the 5′ (CAC TTC TGG TGC TTC TGG C (SEQ ID NO:7)) and 3′ (AAG ATC CGC TAC TTG CTC C (SEQ ID NO:8)) ends to allow amplification and dU cloning (XbaI and XmaI sites).

With the exception of CD20, CD33 and DARC, all proteins were truncated at the N- and/or C-termini to enhance surface expression. The deletions at the N-termini were to remove pre- and pro-sequences (VIPR1 and F2R) and the C-termini deletions were to remove internalization signals (VIPR1, ADORA2A, F2R, EP4, CXCR4, LPAR1, GRPR, and ADRB2). The regions of the proteins used to construct synthetic genes were as follows: region 31-402 of VIPR1 (Uniprot Accession No. P32241); region 1-311 of ADORA2A (Uniprot Accession No. P29274); region 42-377 of F2R (Uniprot Accession No. P25116); region 1-350 of EP4 (Uniprot Accession No. P35408); region 1-318 of CXCR4 (Uniprot Accession No. P61073) with three phosphorylation sites mutated to alanine (T311A, S312A, T318A); region 1-340 of LPAR1 (Uniprot Accession No. Q92633); region 1-343 of GRPR (Uniprot Accession No. P30550); region 1-365 of ADRB2 (Uniprot Accession No. P07550); region 18-364 of CD33 (Uniprot Accession No. P20138); full-length CD20 (Uniprot Accession No. P11826); and full-length DARC (Uniprot Accession No. Q16570). Each of these sequences from the indicated Accession Numbers is incorporated herein by reference.

### Example 3

### Cell Culture and Transfections

FreeStyle™ 293-F cells were obtained from Invitrogen Corporation (Carlsbad, Calif.). Cells were subcultured as outlined by the manufacturer. Briefly, cells were grown in FreeStyle™ 293 Expression Medium (Invitrogen Corp) in 125 mL shaker flasks. Flasks were seeded at a density of 1×10⁵ viable cells/mL (30 mL final volume). Flasks were incubated in a humidified incubator at 37° C., 8% CO₂ on an orbital shaker platform rotating at 130 rpm. Cell density and viability was monitored and cells were subcultured when the density reached 1×10⁶ viable cells/mL.

Twenty-four hours before the transfection, the 293-F cells were subcultured at a density of about 6×10⁵ cells/mL. The day of transfection, the viability of the cells was determined to be >90%, and the cells were diluted to a density of 1×10⁶ cells/mL and 30 mL was placed in each 125 mL shaker flask. The plasmid DNA was diluted as recommended for the FreeStyle™ 293-F cells. Briefly, 37.5 μg of DNA was added to OptiPro™ SFM to a final volume of 0.6 mL and mixed. In a second tube, 37.5 μL of Invitrogen's FreeStyle™ MAX reagent was added to a total OptiPro™ SFM final volume of 0.6 mL and mixed by inversion. The contents of the two tubes were incubated for 5 mins. The two tubes were then mixed and incubated for 30 mins at room temperature. The mixture was added slowly with swirling to the flask containing the cells. The flask was incubated at 37° C., 8% CO₂ on an orbital shaking platform rotating at 130 rpm.

### Example 4

### Flow Cytometry

Flow cytometry was performed on a Guava EasyCyte Plus (Millipore, Billerica, Mass.). Briefly, 2-5×10⁴ transfected cells were placed in each well of a 96 well V bottom plate and stained with saturating amounts of fluorescently-labeled monoclonal antibodies (FITC or phycoerythrin (PE)) Anti-HA (Miltenyi Biotec, Auburn Calif.); Anti-c-Myc (Calbiochem-EMD, San Diego, Calif.); PE-CD20, PE-CD33, and PE-Anti-DARC (R&D Systems, Minneapolis, Minn.); PE-Anti-CXCR4 (Biolegend, San Diego, Calif.); or PE-labeled isotype controls (Santa Cruz Biotechnology, Santa Cruz, Calif.). All staining was in a final volume of 50 μl of 10% normal goat serum (heat inactivated, 30 minutes at 56° C.) in PBS with 0.025% sodium azide and performed at 2-8° C. After 30 minutes with gentle shaking, cells were washed three times with cold 1% bovine serum albumen (BSA) in PBS with 0.025% sodium azide and analyzed. Flow cytometer calibration was performed using Rainbow Calibrator Particles (Spherotech, Lake Forest, Ill.).

For surface staining, only viable cells, as judged by their light scatter characteristics (forward angle and side scatter), were gated to be included in the analysis. Total staining (surface plus internal) was performed using Fix and Perm Cell Permeabilization Kit (Invitrogen, Camarillo, Calif.) as follows: duplicate wells were stained as described previously. After staining and washing, one well of the replicate was fixed using 50 μl of the kit medium A for 30 minutes at room temperature. After washing, cells were resuspended in 50 μl of kit medium B to permeabilize the cells and antibody again added. After staining for 30 minutes at 2-8° C. with gentle shaking, cells were washed with cold 1% BSA in PBS, 0.025% sodium azide and 0.1% saponin to facilitate washing and analyzed, along with the replicate that received only the surface staining

### Example 5

### Results

It was desired to add a tag to membrane proteins that would allow the measurement of their expression level at the plasma membrane, yet not interfere with the structure of the membrane protein. To accomplish this, an expression plasmid was created that fuses an additional transmembrane domain and an epitope tag to the cytoplasmically located C-termini of a membrane protein. Many membrane proteins including GPCRs and ion channels have C-termini located in the cytoplasm. The additional transmembrane domain-epitope tag portion of the construct was designed the “snorkel tag,” as it is designed to project an epitope tag out on the surface of the cell from the cytoplasmically located C-termini. In this way, the epitope tag is separated in space from the extracellular structure of the bound membrane protein.

The required topology of the additional transmembrane domain is N-termini inside, C-termini outside. A transmembrane domain was selected from a type I membrane protein (PDGFRB). Since the PDGFRB transmembrane domain's natural orientation is reversed (e.g., N-termini outside, C-termini inside), the residues flanking the PDGFRB transmembrane domain were altered to conform to the “positive inside” criteria for this vector. The PDGFRB domain has been used previously to create chimeric membrane proteins (Ho et al., 2009, Methods Mol. Biol., 562:99-113), but not with this reversed orientation. In one instance, two epitope tags were added in tandem to the extracellular domain, with the FLAG epitope followed by the HA epitope. Three unique restriction enzyme sites (EcoRI, XbaI, XmaI) were added to the 5′ end of the reading frame to allow the insertion of genes encoding a membrane protein. The resulting plasmid was designated pSNKL-Q. A schematic representation of the snorkel tag protein fused to CD20 is shown in FIG. 1.

The performance and effect of the “snorkel tag” was evaluated using three different multipass membrane proteins; CD20, DARC and CXCR4, which contain four, seven and seven transmembrane domains, respectively. Synthetic genes for CD20, DARC and CXCR4 were cloned into the pSNKL-Q plasmid in-frame to the snorkel tag. The DARC and CXCR4 constructs included a leader sequence and modifications to the C-termini to enhance surface expression (as described above). As controls, constructs for CD20 and DARC were constructed with a stop codon at the 3′ end of the membrane protein to block read through to the “snorkel tag”.

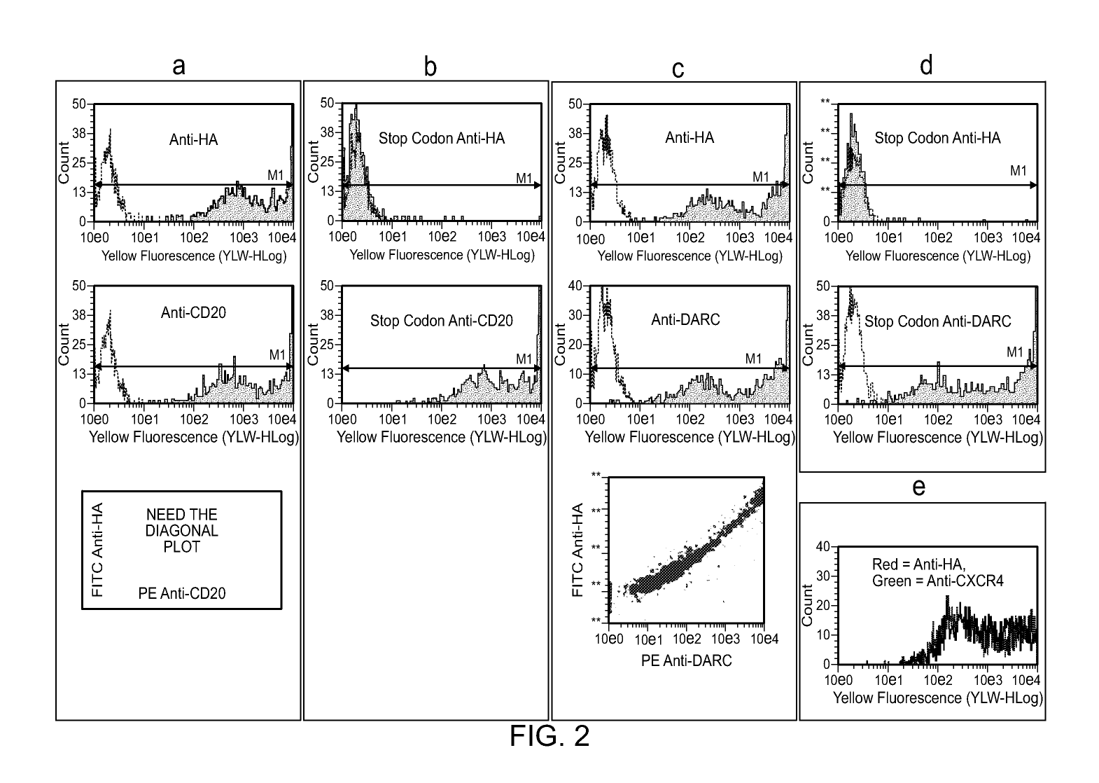

The resulting vectors were transiently transfected into HEK293 cells and the surface expression monitored in flow cytometry, either using CD20-, DARC-, or CXCR4-specific antibodies or an anti-HA epitope tag antibody. The staining pattern and expression levels seen with antibodies against CD20, DARC or CXCR4 compared to antibodies against HA were highly correlated (FIG. 2A, C, E). Moreover, the correlation held up over three orders of magnitude in expression level. Absence of the “snorkel tag” by introducing a stop codon into the CD20 and DARC constructs also showed similar patterns and levels of staining with their cognate antibodies, but, as expected, staining with anti-HA antibodies was eliminated (FIG. 2B, D).

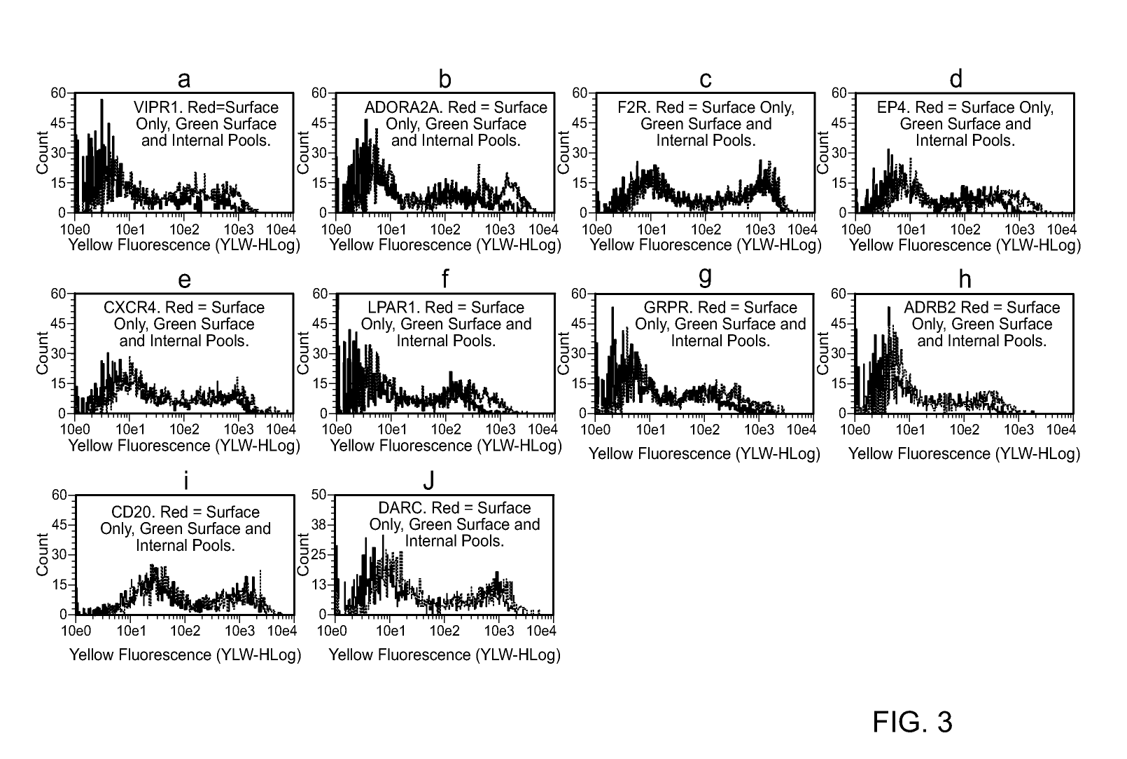

The “snorkel tag” described herein was used to monitor surface expression of a set of ten membrane proteins. All of the membrane proteins used were GPCRs except for CD20. Some of the genes were trimmed at the N- and/or the C-termini to remove sequences that may have limited surface expression as described above. These include pre- and pro-sequences, and internalization signals in the cytoplasmic C-termini. Synthetic genes for VIPR1, ADORA2A, F2R, EP4, CXCR4, LPAR1, GRPR, ADRB2, CD20, and DARC were cloned into the “snorkel vector,” pSNKL-Q, and the vectors were used to transiently transfect HEK293 cells. The level of expression was assessed using flow cytometry with anti-HA antibodies. Since the amount of internalized protein can be evaluated by treating the cells with a permeability agent, flow cytometry also was performed with permeabilized cells. High levels of surface-localized protein were seen for all ten proteins (FIG. 3), some of which had greater staining with permeabilization, indicating that a fraction of the protein was trapped intracellularly (FIG. 3A, B, D, F, G, H).

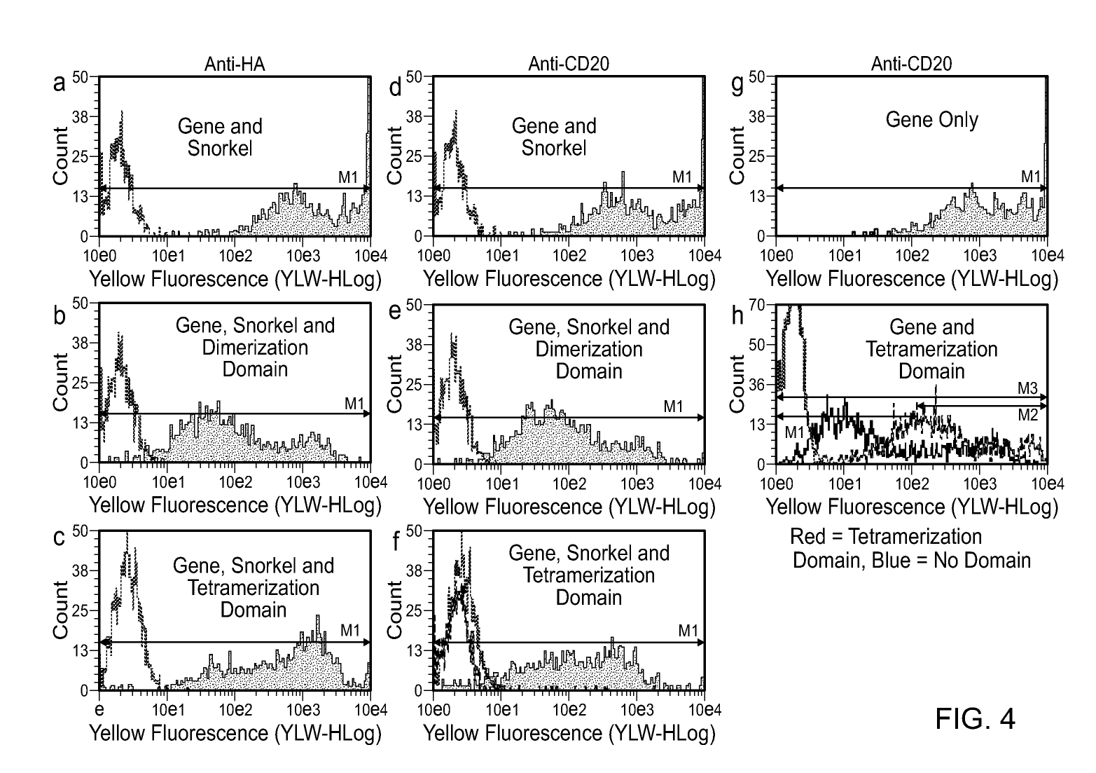

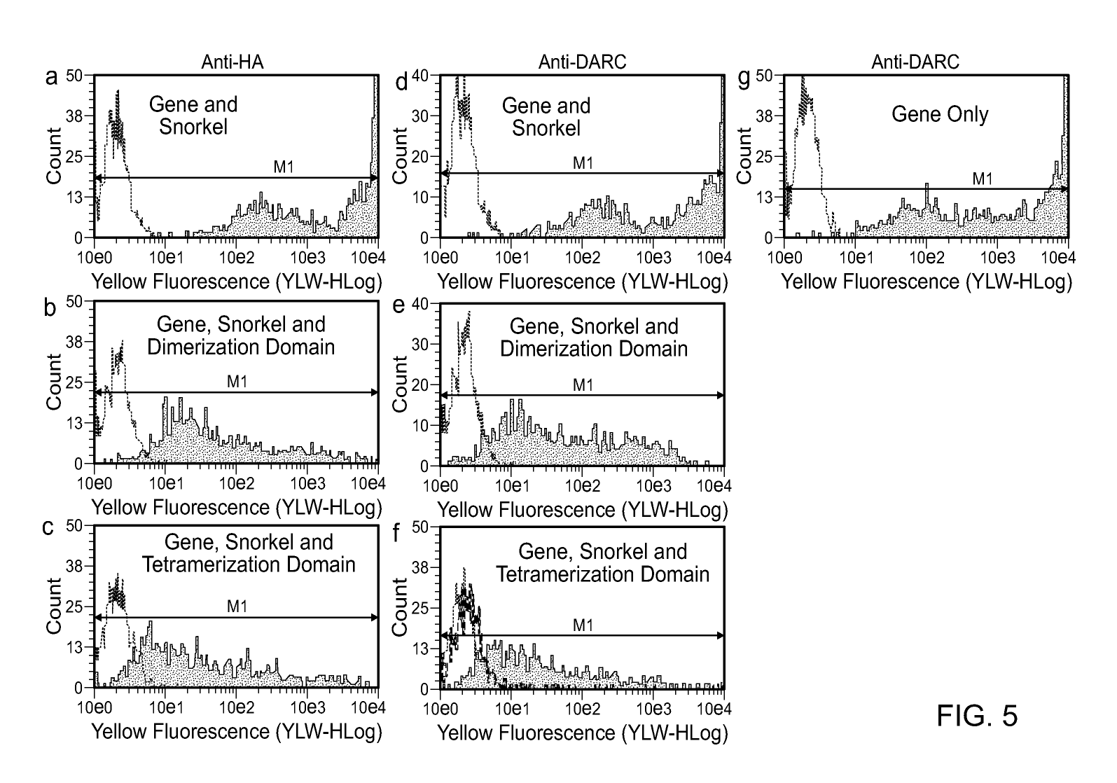

To more rigorously test the reliability of the “snorkel tag” for determining the presence of the target membrane protein, it was applied to situations where the localization may be perturbed. Membrane proteins commonly exist as oligomers and, in at least some instances, have been shown to be required for the delivery of the protein to the surface (Milligan, 2008, J. Pharmacol., 153:S216-29). CD20 and DARC were selected, which had previously been shown to almost exclusively localize to the plasma membrane (FIG. 3I, J). Each gene was used to make four different constructs: gene only; gene and “snorkel tag”; gene, “snorkel tag” and dimerization domain (coiled coil); and gene, “snorkel tag” and tetramerization domain (coiled coil). CD20 also was constructed with the gene and the tetramerization domain but with no “snorkel tag”. The multimerization domains were inserted between the membrane protein and the cytoplasmic side of the “snorkel tag”. Each plasmid was transiently transfected into HEK293 cells and the expression monitored using flow cytometry with membrane protein specific antibodies (anti-CD20 and anti-DARC), and with anti-HA antibodies. As shown in FIG. 2, the pattern and level of expression was very similar for constructs with and without a “snorkel tag” whether stained with anti-HA or anti-CD20 or anti-DARC antibodies (FIG. 4A, D, G; FIG. 5A, D, G). However, there was a significantly lower level of surface expression when either a dimerization or tetramerization domain was included (FIG. 4B, C, E, F; FIG. 5B, C, E, F). Importantly, both the membrane protein antibodies and the HA antibodies showed similar results, indicating that the “snorkel tag” is faithfully reporting on the surface status of the target protein. The lower level of expression with the multimerization domains was also observed when a tetramerization domain was added to CD20 with and without the “snorkel tag” (FIG. 4H). Intracellular staining of the cells revealed that the constructs with multimerization domains had significant amounts of the protein trapped intracellularly (data not shown). Interestingly, the tetramerization domain did not reduce secreted/surface levels of proteins when fused to soluble secreted proteins or single pass membrane proteins.

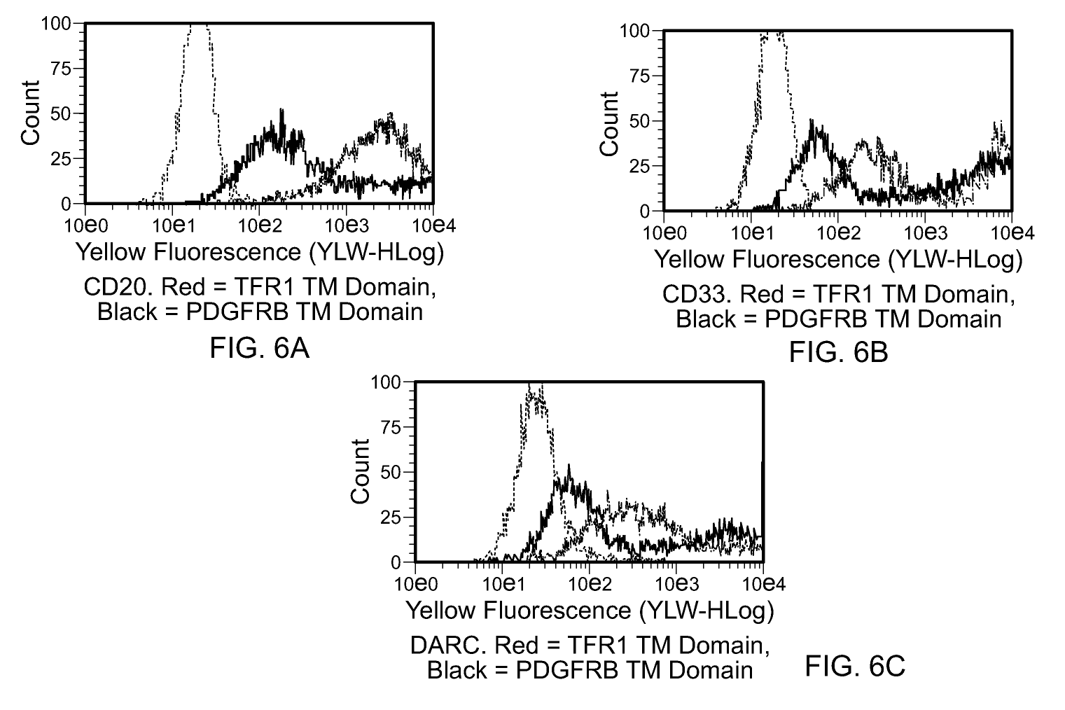

The “snorkel tag” design was based on a transmembrane domain from PDGFRB, a type I membrane protein with the N-terminus on the outside. One concern with the PDGFRB transmembrane domain was that its natural orientation was reversed, with the N-terminus on the inside. Therefore, the performance of the “snorkel tag” was investigated using a different transmembrane domain. This transmembrane domain was from a natural type II membrane protein, mouse transferrin receptor (TRF1), which has the N-terminus on the inside. In order to more sensitively test the performance, this transmembrane domain was evaluated in the context of a tetramerization domain fusion. Three membrane protein genes, CD20, CD33 and DARC, were cloned into the two different “snorkel vectors,” one with the PDGFRB domain and one with the TFR1 domain. The plasmids were transiently transfected into HEK293 cells and stained with anti-HA antibodies. The TFR1 transmembrane domain constructs for all three proteins showed significantly lower surface expression compared to constructs with the PDGFRB transmembrane domain (FIG. 6).

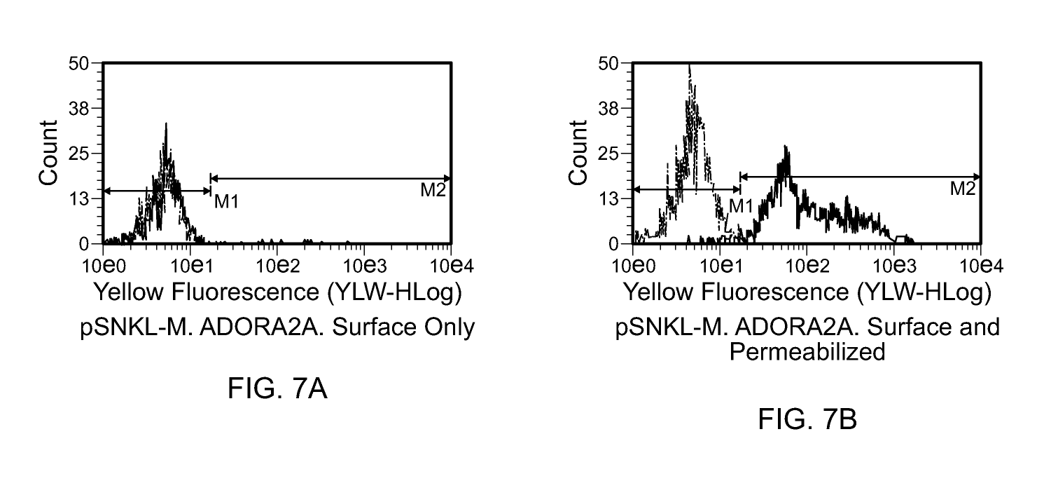

It would be useful to have “snorkel tags” with different epitope tags to allow for experiments to compare different membrane protein constructs in the same cells. Therefore, the entire extracellular domain of pSNKL-Q containing the FLAG and HA tags was switched to one with the Myc tag (pSNKL-M). ADORA2A was selected as a sensitive reporter to test the vector, since it had relatively modest expression levels due to intracellular trapping (FIG. 3B). The ADORA2A gene was cloned into pSNKL-M, the plasmid transiently transfected into HEK293 cells, and the expression monitored with flow cytometry using an anti-Myc antibody. Surprisingly, no Myc tag was detected on the surface of the cells. Furthermore, permeabilization revealed all of the protein was all trapped inside the cells (FIG. 7).

### Example 6

### “Snorkel Tag” with Ion Channels

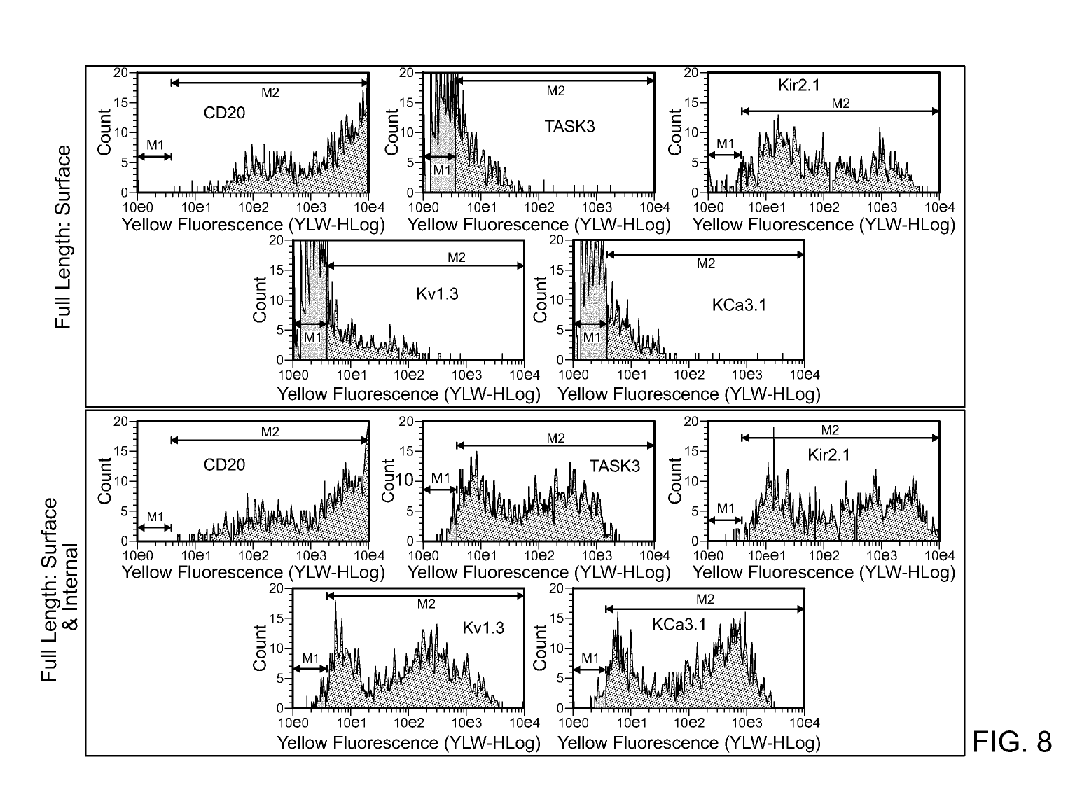

A panel of ion channel genes were cloned into pSNKL-UH3 and evaluated for the expression of the associated HA tag using an anti-HA antibody. The ion channel genes were selected from the potassium channels: TASK3, Kir2.1, Kv1.3, and KCa3.1. Plasmids were transfected into HEK293 cells and evaluated by flow cytometry for both surface and internal staining Significant staining of the HA tag was observed for all of the ion channel plasmids (FIG. 8). These results demonstrated that very few copies of TASK3, KV 1.3 and KCa3.1 are expressed on the surface of the cell and, instead, are trapped inside, whereas almost all of the CD20 and Kir2.1 in a cell are expressed on the surface (FIG. 8). This is consistent with the literature reports on the expression of these proteins.

### Example 7

### “Snorkel Tag” with Different Epitope Tags

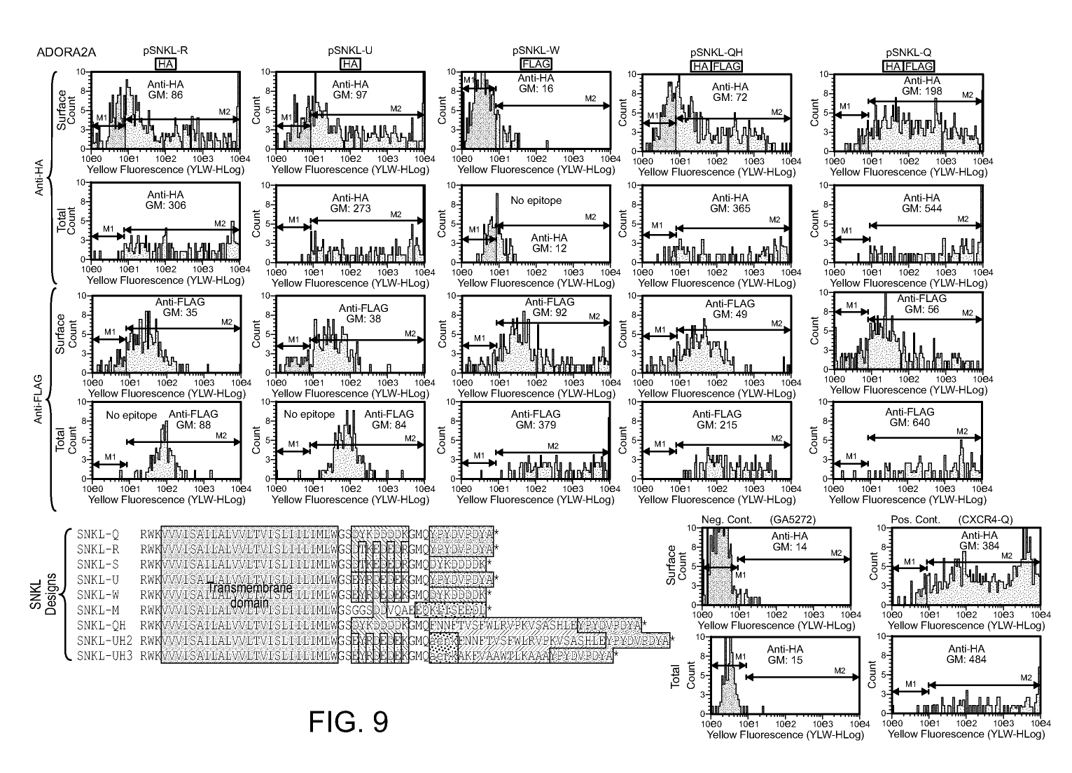

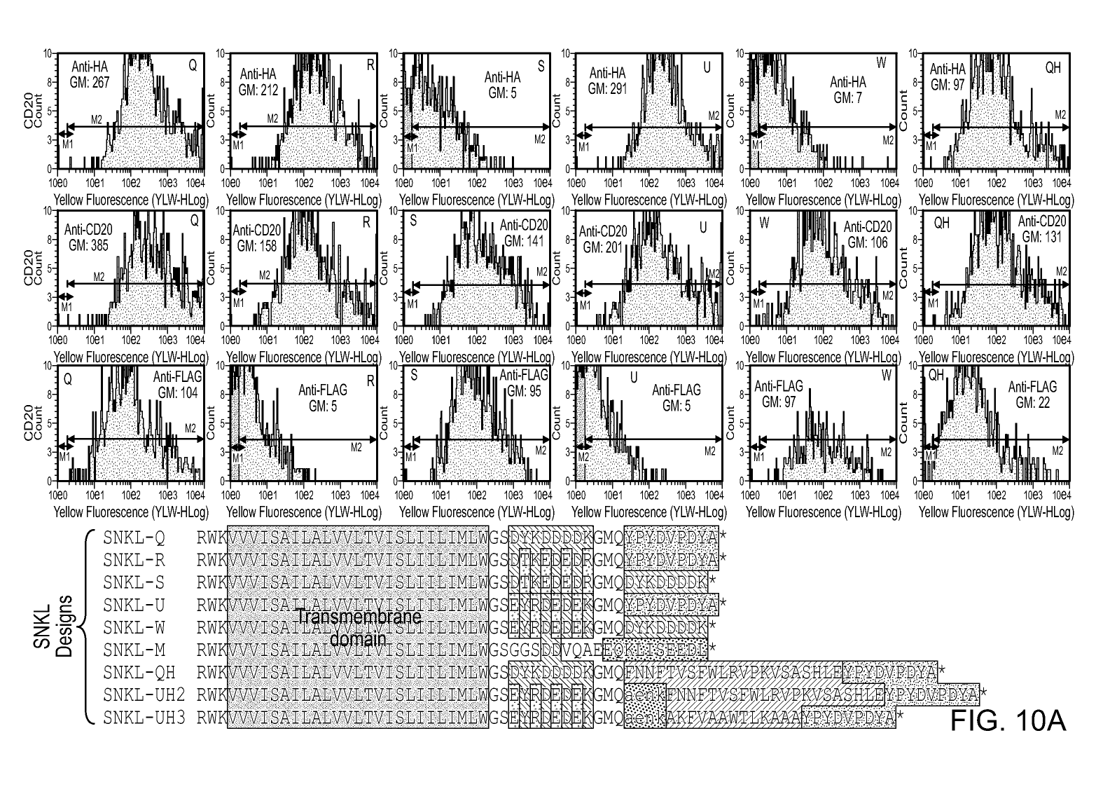

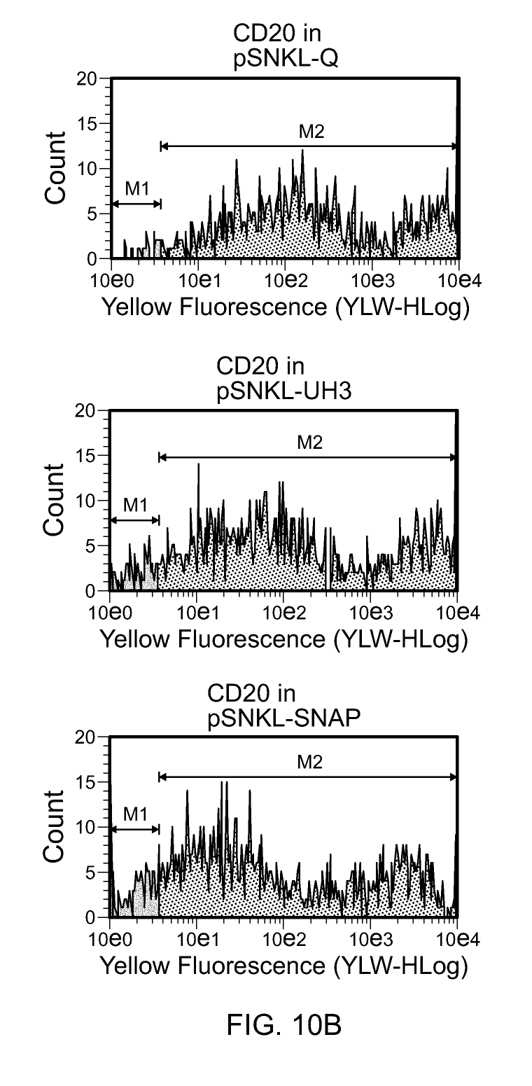

A panel of snorkel plasmids was constructed with different epitope tags at the C-terminus. The original design in pSNKL-Q contained both FLAG and HA tags. Modifications to the design were created in order to have snorkel tags with no common extracellular sequences in order to make them more useful for immunization and subsequent screening experiments (ie immunize with one type and screen on another in order to subtract the signal from antibodies raised to the snorkel tag). In addition, T helper epitopes were added to facilitate immune responses. CD20 and ADORA2A genes were cloned into the various pSNKL plasmids (pSNKL-R, pSNKL-S, pSNKL-U, pSNKL-W, pSNKL-QH) in order to evaluate the performance. CD20 is a highly expressed protein whereas ADORA2A is relatively weakly expressed and significant amounts of protein are trapped intracellularly. Transfected HEK293 cells were stained with anti-HA or anti-FLAG and analysed by flow cytometry. The results showed that all of the snorkel tag variants successfully expressed their respective epitope tags in HEK293 cells with either ADORA2A (FIG. 9) or CD20 (FIG. 10). As expected, vectors with exclusively the HA tag stained with anti-HA antibodies and not anti-FLAG antibodies, and vice versa (see, for example, FIG. 9). For the CD20 constructs, staining with the epitope tag antibodies gave a similar pattern as staining with anti-CD20 antibodies.

To evaluate the capacity of the SNORKEL tag to accommodate larger tags a plasmid was constructed encoding the SNAPtag (Cisbio) to create a final extracellular SNORKEL domain of 204 residues (the largest extracellular domain tested in the previous set was 47 residues in the pSNKL-UH2 plasmid. CD20 was cloned into pSNKL-Q, pSNKL-UH3 and pSNKL-SNAP. The surface expression of CD20 was monitored with a CD20 antibody, which showed similar expression levels (FIG. 10).

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

What is claimed is:

1. A nucleic acid construct, comprising:
a nucleic acid encoding a membrane protein operably linked to a nucleic acid encoding a single membrane-spanning domain operably linked to a nucleic acid encoding a detectable protein,
wherein the C-terminus of the membrane protein is cytoplasmic; and
wherein the single membrane-spanning domain is configured such that the N-terminus of the membrane-spanning domain is intracellular and the C-terminus of the membrane-spanning domain is extracellular.

2. The construct of claim 1, wherein the membrane protein is a G-protein coupled receptor (GPCR) protein, an ion channel protein, or a single pass type I membrane protein.

3. The construct of claim 2, wherein the ion channel protein is selected from the group consisting of a potassium channel protein, a TRP channel protein, a four-domain channel protein, a chloride channel protein, a ligand-gated ion channel protein, a transporter protein, a CD marker protein, a tetraspannin protein, and an aquaporin protein.

4. The construct of claim 1, wherein the membrane protein is selected from the group consisting of VIPR1, ADORA2A, F2R, EP4, CXCR4, LPAR1, GRPR, ADRB2, EAG, Nav1.7, CLCA1, nAChR, ABCA1, SLC5A1, MS4A1, AQP1, CD33, CD20, DARC, Kir2.1, Kir2.2, Kir7.1, Kv10.1, Kv11.1, TASK3, TRPV3, and TRPV3.

5. The construct of claim 1, wherein the single membrane-spanning domain is from a protein selected from the group consisting of PDGFR B.

6. The construct of claim 1, wherein the detectable protein is selected from the group consisting of FLAG, HA, and Myc.

7. The construct of claim 1, wherein the membrane protein is CD20, DARC, or CXCR4; wherein the single membrane-spanning domain is from PDGFRB; and wherein the detectable protein is HA or FLAG.

8. A construct for detecting a membrane-bound protein, comprising, in the 5′ to 3′ direction,
a multiple cloning site (MCS) for in-frame cloning of a nucleic acid encoding a membrane protein, operably linked to
a nucleic acid encoding a single membrane-spanning domain, wherein the single membrane-spanning domain is configured such that its N-terminus is intracellular and its C-terminus is extracellular, operably linked to
a nucleic acid encoding a detectable protein.

9. The construct of claim 8, further comprising a promoter, wherein the promoter is operably linked to the 5′ end of the MCS.

10. The construct of claim 8, wherein the single membrane-spanning domain is from a protein selected from the group consisting of PDGFR B.

11. The construct of claim 8, wherein the detectable protein is selected from the group consisting of FLAG, HA, and Myc.

12. The construct of claim 8, wherein the membrane protein is CD20, DARC, or CXCR4; wherein the single membrane-spanning domain is from PDGFRB; and wherein the detectable protein is HA or FLAG.

13. A nucleic acid construct, comprising:
a nucleic acid encoding a membrane protein operably linked to a nucleic acid encoding a single membrane-spanning domain operably linked to a nucleic acid encoding a detectable protein,
wherein the C-terminus of the membrane protein is cytoplasmic; and
wherein the single membrane-spanning domain is configured such that the N-terminus of the membrane-spanning domain is intracellular and the C-terminus of the membrane-spanning domain is extracellular, wherein the membrane protein is a G-protein coupled receptor (GPCR) protein, an ion channel protein, or a single pass type I membrane protein.

14. A fusion protein, wherein the fusion protein comprises, in the amino- to carboxy-terminal direction, a membrane protein having a cytoplasmic C-terminus, a single membrane-spanning domain having an extracellular C-terminus, and a detectable protein.

15. The fusion protein of claim 14, wherein the membrane protein is a G-protein coupled receptor (GPCR) protein, an ion channel protein, or a single pass type I membrane protein.

16. The fusion protein of claim 15, wherein the ion channel protein is selected from the group consisting of a potassium channel protein, a TRP channel protein, a four-domain channel protein, a chloride channel protein, a ligand-gated ion channel protein, a transporter protein, a CD marker protein, a tetraspannin protein, and an aquaporin protein.

17. The fusion protein of claim 14, wherein the membrane protein is selected from the group consisting of VIPR1, ADORA2A, F2R, EP4, CXCR4, LPAR1, GRPR, ADRB2, EAG, Nav1.7, CLCA1, nAChR, ABCA1, SLC5A1, MS4A1, AQP1, CD33, CD20, DARC, Kir2.1, Kir2.2, Kir7.1, Kv10.1, Kv11.1, TASK3, TRPV3, and TRPV3.

18. The fusion protein of claim 14, wherein the single membrane-spanning domain is heterologous to the membrane protein.

19. The fusion protein of claim 14, wherein the single membrane-spanning domain is from a protein selected from the group consisting of PDGFR B.

20. The fusion protein of claim 14, wherein the detectable protein is selected from the group consisting of FLAG, HA, and Myc.

21. The fusion protein of claim 14, wherein the membrane protein is CD20, DARC, or CXCR4; wherein the single membrane-spanning domain is from PDGFRB; and
wherein the detectable protein is HA or FLAG.

22. A method of expressing a fusion protein, comprising the steps of:
culturing a recombinant host cell comprising the construct of claim 1 under conditions that promote the expression of the fusion polypeptide.

23. A method of detecting a membrane protein bound to the membrane of a cell, comprising the steps of:
(a) culturing a recombinant host cell that comprises a nucleic acid molecule that encodes the fusion protein of claim 14 under conditions suitable for expression of the fusion polypeptide; and
(b) detecting the detectable protein portion of the fusion protein bound to the cell, thereby detecting the membrane protein bound to the membrane of the cell.

24. The method of claim 23, wherein the host cell is a eukaryotic host cell.

25. The method of claim 24, wherein the eukaryotic host cell is selected from the group consisting of HEK-293 cells, CHO cells, Swiss 3T3 cells, and yeast cells.

26. The method of claim 23, wherein the detecting step utilizes a labeled antibody that binds to the detectable protein.

27. A method of quantitating the amount of a membrane protein bound to the membrane of a cell, comprising the steps of:
(a) culturing a recombinant host cell that comprises a nucleic acid molecule that encodes the fusion protein of claim 14 under conditions suitable for expression of the fusion polypeptide; and
(b) quantitating the amount of detectable protein bound to the cell, thereby quantitating the amount of the membrane protein bound to the membrane of the cell.

28. The method of claim 27, wherein the host cell is a eukaryotic host cell.

29. The method of claim 28, wherein the eukaryotic host cell is selected from the group consisting of HEK-293 cells, CHO cells, Swiss 3T3 cells, and yeast cells.

30. The method of claim 27, wherein the quantitating step comprises contacting the cell with a labeled antibody that binds to the detectable protein and determining the amount of labeled antibody that bound.
